(19) United States
(12) Patent Application Publication
(10) Pub. No.: US 2024/0156852 A1
(43) Pub. Date: May 16, 2024

(54) CARBON NANOPARTICLE COMPOSITIONS AND METHODS FOR DELIVERING THERAPEUTICS TO SPECIFIC TARGET SITES

(71) Applicant: SINON NANO SCIENCES INC., Durham, NC (US)

(72) Inventors: Sabyasachi Sarkar, Shibpur (IN); Afreen Allam, Durham, NC (US); Arsheen Allam, Durham, NC (US); Abdul Allam, Durham, NC (US)

(21) Appl. No.: 18/284,278

(22) PCT Filed: Mar. 10, 2022
(86) PCT No.: PCT/US22/19688

§ 371 Date: Sep. 26, 2023

Related U.S. Application Data
(60) Provisional application No. 63/159,858, filed on Mar. 11, 2021.

(51) Int. Cl. A61K 31/713; A61K 9/51; A61K 31/337; A61K 33/44

(52) U.S. Cl. CPC A61K 31/713; A61K 9/5192; A61K 31/337; A61K 33/44

(57) ABSTRACT

Described herein are nanoparticle compositions and methods for pH-specific release and targeted delivery of therapeutics with enhanced bioavailability. In some embodiments, methods are described for generating carbon nanoparticles (CNPs) that can release payload in acidic pH environments.

### CROSS-REFERENCE TO RELATED APPLICATION(S)

This application claims priority to U.S. Provisional Patent Application No. 63/159,858, filed on Mar. 11, 2021, which is incorporated by reference herein in its entirety.

### TECHNICAL FIELD

Described herein are nanoparticle compositions and methods for pH-specific release and targeted delivery of therapeutics with enhanced bioavailability. In some embodiments, methods are described for generating carbon nanoparticles (CNPs) that can release payload in acidic pH environments.

### BACKGROUND

Nanocarriers provide new drug delivery methods for the treatment of neurological disorders, cardiovascular disorders, and treatment of various forms of cancer. A nanocarrier is nanomaterial used as a transport module for another substance, such as a drug, biomolecule, mRNA, gene, etc. Commonly used nanocarriers include micelles, polymers, carbon-based materials, liposomes, and other substances.

There is a growing trend towards use of nanocarriers to encapsulate chemotherapeutic agents to be delivered to disease areas such as tumors. Various nanocarriers have been approved in clinical cancer chemotherapy and have shown improvement in therapeutic efficiency compared to traditional formulations, such as liposomes (e.g., Doxil®, Lipusu®), nanoparticles (e.g., Abraxane®), and micelles (e.g., Genexol-PM®).

It is widely accepted that the pH (extracellular pH) of cancer cells is more acidic than normal cells. Generally, pH values of normal tissues (e.g., brain tissues, subcutaneous tissues, etc.) are in the pH range of 7.2-7.5. However, the pH of tumor cells is mildly acidic in the range of 6.4-7.0. This acidic nature of tumor cells can be exploited and used for targeted drug delivery and for the release of drug payload in this specific pH range.

Thus, it would be beneficial to provide nanoparticle compositions that are pH-sensitive for targeted drug delivery and release, and methods of producing the pH-sensitive nanoparticles.

### SUMMARY

One embodiment described herein is a composition comprising graphene oxide nanoparticles (CNP) comprising a plurality of graphene sheets having a plurality of carboxylic acid groups and hydroxyl groups and interconvertible open and closed forms; the open form having a size of about 40 nm to 200 nm in breadth and length in open sheet form; the closed form having a diameter of about 50 nm to 80 nm; wherein the closed form can be converted to the open form at a pH of about 6.8 or lower; and the open form can be converted to the closed form at a pH of about 7.0 or greater. In one aspect, the carboxylic acid groups comprise at least about 25-35% of the total mass of the CNP. In another aspect, the hydroxyl groups comprise at least about 15% of the total mass of the CNP. In another aspect, the CNP has a solubility in aqueous solution at a concentration of about 1 mg CNP/mL. In another aspect, the CNP displays fluorescence in the blue, green, red, and infrared spectra. In another aspect, the CNP has amphiphilic properties.

Another embodiment described herein is a method for reversible encapsulation of a molecule within a CNP, the method comprising: contacting the open form CNP with a molecule to be encapsulated; converting the open form CNP to the closed form CNP with the molecule encapsulated therein by adjusting the pH to about 7.0 or greater; and releasing the encapsulated molecule from the closed form CNP by converting back to the open form CNP at a pH of about 6.8 or lower. In one aspect, the molecule is one or more of a small molecule pharmaceutical, a protein, a peptide, a nucleic acid, a single strand DNA, a double strand DNA, an RNA, an siRNA, an oligonucleotide, a gene, a gene fragment, an imaging agent, a lanthanide, or a combination thereof. In another aspect, the molecule is no more than about 500 kDa in size.

Another embodiment described herein is a composition comprising a water-soluble carbon nanoparticle (CNP) having an encapsulated molecule therein, the CNP comprising a plurality of graphene sheets having a plurality of carboxylic acid groups and hydroxyl groups and interconvertible open and closed forms; the open form having a size of about 40 nm to 200 nm in breadth and length in open sheet form; the closed form having a diameter of about 50 nm to 80 nm; wherein the closed form can be converted to the open form at a pH of about 6.8 or lower; and the open form can be converted to the closed form at a pH of about 7.0 or greater. In one aspect, the carboxylic acid groups comprise at least about 25-35% of the total mass of the CNP. In another aspect, the hydroxyl groups comprise at least about 15% of the total mass of the CNP. In another aspect, the encapsulated molecule is an imaging agent or a therapeutic agent. In another aspect, the encapsulated molecule is a therapeutic agent for the treatment of cancer. In another aspect, the encapsulated molecule is one or more of a small molecule pharmaceutical, a protein, a peptide, a nucleic acid, a single strand DNA, a double strand DNA, an RNA, an siRNA, an oligonucleotide, a gene, a gene fragment, an imaging agent, a lanthanide, or a combination thereof. In another aspect, the molecule is no more than about 500 kDa in size. In another aspect, the composition further comprises one or more biomolecules or divalent metals for targeted delivery of the CNP with the encapsulated molecule to a cell, tissue, brain, or organ. In another aspect, the biomolecule is a protein, a receptor, an aptamer, a ligand, or an antibody; and the divalent metal is manganese (Mn).

Another embodiment described herein is a method for treating a subject with cancer, the method comprising: delivering to the subject a pH-sensitive water-soluble carbon nanoparticle (CNP) having an encapsulated molecule therein, wherein the CNP comprises a plurality of graphene sheets having a plurality of carboxylic acid groups and hydroxyl groups and interconvertible open and closed forms; the open form having a size of about 40 nm to 200 nm in breadth and length in open sheet form; the closed form having a diameter of about 50 nm to 80 nm; wherein the closed form can be converted to the open form at a pH of about 6.8 or lower; and the open form can be converted to the closed form at a pH of about 7.0 or greater; and wherein the encapsulated molecule is released from the CNP at a pH of 6.8 or lower after delivery to the subject. In one aspect, delivering comprises parenteral administration, oral administration, or inhalation. In another aspect, the molecule is one or more of a small molecule pharmaceutical, a protein, a peptide, a nucleic acid, a single strand DNA, a double strand DNA, an RNA, an siRNA, an oligonucleotide, a gene, a gene fragment, an imaging agent, a lanthanide, or a combination thereof. In another aspect, the small molecule pharmaceutical is paclitaxel. In another aspect, the cancer is breast, ovarian, lung, bladder, prostate, melanoma, esophageal, stomach, other solid tumor cancers, combinations thereof. In another aspect, the subject has glioblastoma. In another aspect, the CNP further comprises one or more biomolecules or divalent metals for targeted delivery of the CNP with the encapsulated molecule to a cell, tissue, organ, or organ system. In another aspect, wherein the biomolecule is a protein, a receptor, an aptamer, a ligand, or an antibody; and the divalent metal is manganese (Mn). In another aspect, the molecule is delivered across the blood brain barrier (BBB) of the subject.

Another embodiment described herein is a method for producing a pH-sensitive water-soluble carbon nanoparticle (CNP), the method comprising: treating a material comprising one or a combination of wood, charcoal, low grade coal, or carbonized plant biomass with a dilute acidic solution to form a mixture of components including insoluble material and a second solution; separating the second solution from the insoluble material; neutralizing the second solution to form a precipitate; and separating the precipitate from the neutralized second solution; wherein the precipitate comprises the CNP, the CNP comprising a plurality of graphene sheets having a plurality of carboxylic acid groups and hydroxyl groups and interconvertible open and closed forms; the open form having a size of about 40 nm to 200 nm in breadth and length in open sheet form; the closed form having a diameter of about 50 nm to 80 nm; wherein the closed form can be converted to the open form at a pH of about 6.8 or lower; and the open form can be converted to the closed form at a pH of about 7.0 or greater. In one aspect, the dilute acidic solution is diluted HNO₃. In another aspect, the carboxylic acid groups comprise at least about 25-35% of the total mass of the CNP. In another aspect, the hydroxyl groups comprise at least about 15% of the total mass of the CNP.

### DESCRIPTION OF THE DRAWINGS

FIG. 1 shows a scanning electron microscopy (SEM) image of an open form of the carbon nanoparticles (CNPs) at pH 6.5.

FIG. 2 shows an SEM image of a closed form of the CNPs at pH 7.5.

FIG. 3 shows a UV-Visible spectrum of the CNP in water. The spectrum has a single broad absorption peak around 267 nm, characteristic of electron transition in the polyaromatic groups of graphene oxide.

FIG. 4A-B show attenuated total reflection infrared (ATR-IR) spectra. FIG. 4A shows spectra of CNP and washed CNP. Broad peaks at 3272 cm⁻¹ and 2981 cm⁻¹ correspond to the O—H stretching of hydroxyl group and C—H stretching of graphene sheet, respectively. Presence of carboxylic group is confirmed by the peak at 1709 cm⁻¹, which correspond to C═O stretching in COOH. FIG. 4B shows a zoomed-in ATR-IR spectra showing the presence of the acid functional group. Peaks corresponding to C═C stretching, O—H bending and C—O stretching are assigned at 1569 cm⁻¹, 1369 cm⁻¹, and 1293 cm⁻¹. Peak at 1077 cm⁻¹ confirms the presence of the epoxy group.

FIG. 5 shows a Raman spectrum of the CNP with a strong peak at 1577 cm⁻¹ corresponding to the in-plane stretching vibration of the sp 2 C—C bonds (G band) within the ordered graphitic layers. Another vibration at 1356 cm⁻¹ (D band) is related to defects in the graphene structure of the CNP. The Raman spectrum confirms the presence of graphene oxide (GO) and reduced graphene oxide (rGO).

FIG. 6 shows a Brunauer-Emmett-Teller (BET) surface area analysis of the CNP.

FIG. 7A-B show powder X-ray diffraction of CNP. CNP were washed in two different ways to remove NaCl completely and recorded PXRD. The two PXRD patterns (FIG. 7A) and (FIG. 7B) of washed CNP show that both samples are amorphous. The peaks at 2θ=24.8 (FIG. 7A) and 24.9 (FIG. 7B) appears due to the hkl plane=(002) and the peaks at 2θ=41.9 (FIG. 7A) and 42.1 (FIG. 7B) are for hkl plane=(100) respectively. These two type peaks reveal reduced graphene oxide peaks.

FIG. 8A-B show dynamic light scattering (DLS) results illustrating the size distribution of the CNPs at pH 7.0, where the CNPs are predominantly in the closed form. FIG. 8A shows a histogram representation of the hydrodynamic radii (nm). FIG. 8B shows the percent distribution of the CNPs by size.

FIG. 9 shows a Zeta potential for the CNP at pH 7 with a value of −34.4 mV. This corresponds to the charge present in the particle when dissolved in water. The −ve indicates that the carboxylic (—COO⁻) and the hydroxyl (—O⁻) groups are responsible. The high negative value also suggests that the CNP is electrostatically stable at pH 7 and does not agglomerate.

FIG. 10A-D show standard curves for paclitaxel in ethanol (EtOH) (FIG. 10A-B) and dichloromethane (DCM) solvents (FIG. 10C-D). FIG. 10A shows UV-Vis spectra of paclitaxel at varying concentrations in EtOH. FIG. 10B shows a standard curve for absorbance (228 nm) vs. concentration of paclitaxel in EtOH. FIG. 10C shows UV-Vis spectra of paclitaxel at varying concentrations in DCM. FIG. 10D shows a standard curve for absorbance (230 nm) vs. concentration of paclitaxel in DCM.

FIG. 11A-B show absorption kinetic studies of paclitaxel in ethanol at 25° C. FIG. 11A shows UV-Vis spectra of paclitaxel in the presence of CNP over different time intervals. FIG. 11B shows a plot of the concentration of paclitaxel vs. time.

FIG. 12 shows a paclitaxel leaching study. UV-Vis was used to monitor paclitaxel released over two cycles of washing with EtOH.

FIG. 13A-C show paclitaxel release profiles by UV-Vis spectra at pH 6.5 (FIG. 13A), pH 6.8 (FIG. 13B), and pH 7.0 (FIG. 13C). FIG. 13D shows release as a function of pH over time.

FIG. 13E shows a histogram diagram of the paclitaxel pH release profile at 72 hr.

FIG. 14 shows a pharmacokinetic study of paclitaxel or CNP loaded with paclitaxel over 24 hours in Wistar rats.

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

The compositions described herein may be administered orally, parenterally, by inhalation spray, topically, rectally, nasally, buccally, vaginally or via an implanted reservoir. The term “parenterally” or “parenteral administration” as used herein includes subcutaneous, intravenous, intramuscular, intra-articular, intra-synovial, intrasternal, intrathecal, intrahepatic, intralesional, intracranial injection, or via infusion. In some embodiments, the compositions described herein are administered orally, intravenously, or by inhalation.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

### CNP Compositions and Methods of Production

Described herein are compositions of graphene oxide nanoparticles (CNPs) that are originally extracted from source organic waste, coke, coal source, etc. The compositions comprise CNPs that vary in size ranging from about 40 nm to about 200 nm in the open form. Each of these CNPs have a different number of functional hydrophilic groups attached, including carbonyls, hydroxyl, and/or carboxylic acid moieties. These CNPs with varying size and functional groups react differently when treated with alkali or base solutions to neutralize the peripheral functional groups. The way in which they react depends on the difference in the pKₐ values of the carboxylic acid groups, as there will be several pKₐ values due to the presence of varying amounts of carboxylic acid groups. However, it is possible to use a specific pH to separate CNPs with different pKₐ values from the bulk mixture of CNPs.

The described methods allow for the preparation of CNPs which “open” and “close” at specific pH values. The specific required pH is used in the production of the CNP having the ability to both open and close. Treatment of CNP mixture with alkali results in creating soluble form of CNP that is in the open form. This solubilized form of CNP is centrifuged and the filtrate is treated to a preferential pH, which will result in separating a specific form of CNP that is susceptible to that specific pH. Acidification of the filtrate obtained on treating CNP mixture with NaOH, is performed by slowly treating it with dilute HCL achieve a pH level of 6.5. This acidification process isolates CNPs that open at preferred pH of 6.5 from the rest of the CNP mixture.

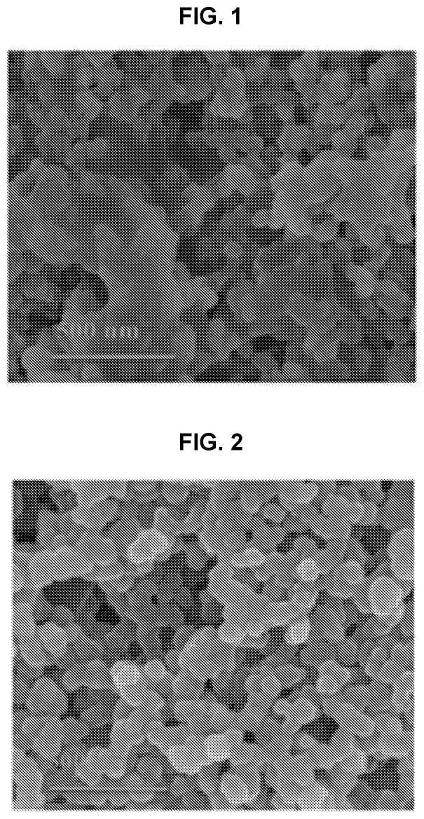

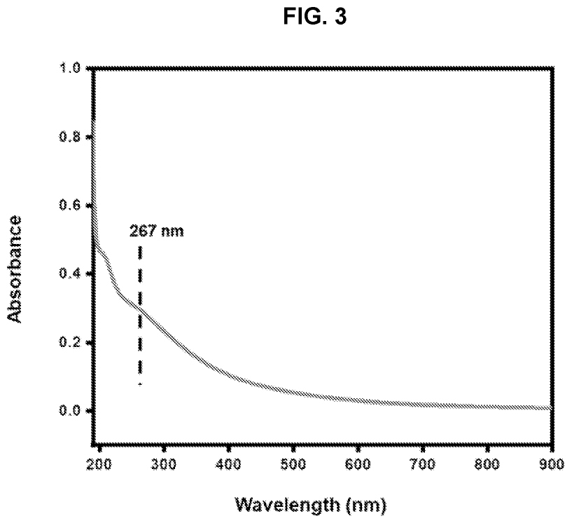

FIG. 1 and FIG. 2 show SEM images confirming the open and closed forms of CNPs, respectively. To confirm the open state of CNPs, 1 mg of CNP is treated with ddH₂O (distilled water) at pH 6.5. This is sonicated for 2-3 minutes. A drop of this solution is used for SEM analysis to show the open state of CNP (FIG. 1). To confirm the closed state of CNPs, 1 mg of CNP is treated with ddH₂O at pH 7.5. This is sonicated for 2-3 minutes. A drop of this solution is used for SEM analysis to show the closed state of CNP (FIG. 2).

The CNP closing (e.g., encapsulation of payload) and opening (e.g., release of payload) is based on the density of carboxylic acid moieties. When the CNP is in open form (flat) the carboxylic acid groups and adjacent hydroxyl groups are side by side. A change in the pH ionizes the carboxylic acid groups to hydrogen bind with adjacent hydroxyl groups. Such hydrogen bond formation will cause the flat CNP (“open form”) to curve leading to the closure of the CNP (“closed form”). This open-close chemistry is thus driven by two parameters: (1) the number of —COOH and/or —OH groups, i.e., the density per unit mass of the CNP and (2) deprotonation of the —COOH group at a particular pH. Thus, the open-close structure of CNP depends on the number of the —COOH and/or —OH groups and on the pH.

The percentage of the —COOH and/or —OH groups can be evaluated by acid-base titrimetry to evaluate the percentage of such groups introduced per unit mass of the CNP. These groups are introduced by chemical reactions taking place under oxidation. In such reaction any variations in treatment time or concentration of oxidizing acid (HNO₃ in this case) will control the density of these functional groups introduced. In order to determine the amount of these groups, standard acid-base titration using a suitable acid-base indicator can be used determine the density of carboxylic acid per unit mass of the CNP. Normally a standard solution of sodium hydroxide is used for titration with methyl orange or phenolphthalein as indicators.

Titration studies have shown that the CNP described herein contain 30%±3 carboxylic acid moieties by mass and approximately 15% of hydroxyl groups by mass. This constitutes a total mass percentage of approximately 45% by mass and the remaining ˜55% by mass is hydrocarbon (predominantly carbon mass).

Therefore, the solubility of the CNP in water or in a buffer is based on the ionization of the number of carboxylic acid groups. The acidic (—COOH) groups in the CNP are added during the chemical oxidation process. The number of —COOH groups per unit area affects the pH where opening/closing will occur. CNPs with more carboxylic acid groups protonate at lower pHs and open the CNP at such pH values.

The CNP composition disclosed herein is distinguished from that disclosed in U.S. Pat. No. 10,988,385, which is incorporated by reference herein for such teachings. The previously described CNPs open under slightly basic pH (e.g., pH 7.4) and close around neutral pH. In contrast, the CNPs described herein open at acidic pH and close at basic pH. The CNP compounds described herein have approximately 27-33% carboxylic acid groups per unit mass as compared to the previous CNPs described in U.S. Pat. No. 10,988,385 which have ˜20%±3 carboxylic acid groups per unit mass. It is noteworthy that an ˜8-13% change in the amount of carboxylic acid moieties caused the pH open/closing profiles to change for these compounds. In addition, the CNPs described herein comprise about 15% of hydroxyl moieties by mass.

The CNPs described herein have an area in the open form of about 40 nm to 200 nm in breadth and length based on SEM studies (e.g., “a composition comprising graphene oxide nanoparticles (CNP) comprising a plurality of graphene sheets having a plurality of carboxylic acid groups and hydroxyl groups and interconvertible open and closed forms; the open form having a size of about 40 nm to 200 nm in breadth and length in open sheet form . . . ”). SEM studies were performed in vacuo using samples at either pH 6.8 or 7.5. However, in solution at pH 7.0 the same CNPs in the closed form have hydrodynamic sizes of about 70 nm to 200 nm as determined by dynamic light scattering. This is due to DLS study being performed in solution at pH 7, where the CNPs are predominantly in the closed form. See e.g., FIGS. 1-2 and 8A-8B.

One embodiment described herein is a composition comprising graphene oxide nanoparticles (CNPs) comprising a plurality of graphene sheets having a plurality of carboxylic acid groups and hydroxyl groups and interconvertible open and closed forms; the flat open form with varying size of about 40 nm to 200 nm in breadth and length in open sheet form based on SEM; on closing to form a round shape having a diameter ranging from 50 nm to 80 nm; wherein the closed form can be converted to the open form at a pH of about 6.8 or lower; and the open form can be converted to the closed form at a pH of about 7.0 or greater. In one aspect, the carboxylic acid groups comprise around 30% (e.g., 25-35%, including all integers within the specified range) of the total mass of the CNP. In another aspect, the hydroxyl groups comprise around 15% of the total mass of the CNP. In another aspect, both versions of CNPs are freely soluble in water. For example, an aqueous solution of 1 mg/mL of CNPs is useful for fluorescence and biological studies. In another aspect, the CNPs described herein display fluorescence in the blue, green, red, and infrared spectra. In another aspect, the CNPs described herein have amphiphilic properties.

Another embodiment described herein is a method for reversible encapsulation of a molecule within a CNP, the method comprising: contacting the open form CNP with a molecule to be encapsulated; converting the open form CNP to the closed form CNP with the molecule encapsulated therein by adjusting the pH to about 6.5 or greater; and releasing the encapsulated molecule from the closed form CNP by converting back to the open form CNP at a pH of about 6.8 or lower. In one aspect, the molecule is one or more of a small molecule pharmaceutical, a protein, a peptide, a nucleic acid, a single strand DNA, a double strand DNA, an RNA, an siRNA, an oligonucleotide, a gene, a gene fragment, an imaging agent, a lanthanide, or a combination thereof. In another aspect, the molecule is no more than about 500 kDa in size.

Another embodiment described herein is a composition comprising a water-soluble carbon nanoparticle (CNP) having an encapsulated molecule therein, the CNP comprising a plurality of graphene sheets having a plurality of carboxylic acid groups and hydroxyl groups and interconvertible open and closed forms; the flat open form with varying size of about 40 nm to 200 nm in breadth and length in open sheet form based on SEM; the closed form having a diameter of about 50 nm to 80 nm; wherein the closed form can be converted to the open form at a pH of about 6.8 or lower; and the open form can be converted to the closed form at a pH of 7.0 or greater. In one aspect, the carboxylic acid groups comprise at least about 25-35% of the total mass of the CNP. In another aspect, the hydroxyl groups comprise at least about 15% of the total mass of the CNP. In another aspect, the encapsulated molecule is an imaging agent or a therapeutic agent. In another aspect, the encapsulated molecule is a therapeutic agent for the treatment of cancer. In another aspect, the encapsulated molecule is one or more of a small molecule pharmaceutical, a protein, a peptide, a nucleic acid, a single strand DNA, a double strand DNA, an RNA, an siRNA, an oligonucleotide, a gene, a gene fragment, an imaging agent, a lanthanide, or a combination thereof. In another aspect, the molecule is no more than about 500 kDa in size. In another aspect, the composition further comprises one or more biomolecules or divalent metals for targeted delivery of the CNP with the encapsulated molecule to a cell, tissue, brain, or organ. In another aspect, the biomolecule is a protein, a receptor, an aptamer, a ligand, or an antibody; and the divalent metal is manganese (Mn).

Another embodiment described herein is a method for treating a subject with cancer, the method comprising: delivering to the subject a pH-sensitive water-soluble carbon nanoparticle (CNP) having an encapsulated molecule therein, wherein the CNP comprises a plurality of graphene sheets having a plurality of carboxylic acid groups and interconvertible open and closed forms; the open form having a size of about 40 nm to 200 nm in breadth and length in open sheet form based on SEM; the closed form having a diameter of about 50 nm to 80 nm; wherein the closed form can be converted to the open form at a pH of about 6.8 or lower; and the open form can be converted to the closed form at a pH of about 7.0 or greater; and wherein the encapsulated molecule is released from the CNP at a pH of 6.8 or lower after delivery to the subject. In one aspect, delivering comprises parenteral administration, oral administration, or inhalation. In another aspect, the molecule is one or more of a small molecule pharmaceutical, a protein, a peptide, a nucleic acid, a single strand DNA, a double strand DNA, an RNA, an siRNA, an oligonucleotide, a gene, a gene fragment, an imaging agent, a lanthanide, or a combination thereof. In another aspect, the small molecule pharmaceutical is paclitaxel. In another aspect, the cancer is breast, ovarian, lung, bladder, prostate, melanoma, esophageal, stomach, other solid tumor cancers, combinations thereof. In another aspect, the subject has glioblastoma. In another aspect, the CNP further comprises one or more biomolecules or divalent metals for targeted delivery of the CNP with the encapsulated molecule to a cell, tissue, organ, or organ system. In another aspect, the biomolecule is a protein, a receptor, an aptamer, a ligand, or an antibody; and the divalent metal is manganese (Mn). In another aspect, the molecule is delivered across the blood brain barrier (BBB) of the subject.

Another embodiment described herein is a method for producing a pH-sensitive water-soluble carbon nanoparticle (CNP), the method comprising: treating a material comprising one or a combination of wood, charcoal, low grade coal, or carbonized plant biomass with a dilute acidic solution to form a mixture of components including insoluble material and a second solution; separating the second solution from the insoluble material; neutralizing the second solution to form a precipitate; and separating the precipitate from the neutralized second solution; wherein the precipitate comprises the CNP, the CNP comprising a plurality of graphene sheets having a plurality of carboxylic acid groups and hydroxyl groups and interconvertible open and closed forms; the open form having a size of about 40 nm to 200 nm in breadth and length in open sheet form based on SEM; the closed form having a diameter of about 50 nm to 80 nm; wherein the closed form can be converted to the open form at a pH of about 6.8 or lower; and the open form can be converted to the closed form at a pH of about 7.0 or greater. In one aspect, the dilute acidic solution is diluted HNO₃. In another aspect, the carboxylic acid groups comprise at least about 25-35% of the total mass of the CNP. In another aspect, the hydroxyl groups comprise at least about 15% of the total mass of the CNP.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A composition comprising graphene oxide nanoparticles (CNP) comprising a plurality of graphene sheets having a plurality of carboxylic acid groups and hydroxyl groups and interconvertible open and closed forms; the open form having a size of about 40 nm to 200 nm in breadth and length in open sheet form; the closed form having a diameter of about 50 nm to 80 nm; wherein the closed form can be converted to the open form at a pH of about 6.8 or lower; and the open form can be converted to the closed form at a pH of about 7.0 or greater.
Clause 2. The composition of clause 1, wherein the carboxylic acid groups comprise at least about 25-35% of the total mass of the CNP.
Clause 3. The composition of clause 1 or 2, wherein the hydroxyl groups comprise at least about 15% of the total mass of the CNP.
Clause 4. The composition of any one of clauses 1-3, wherein the CNP has a solubility in aqueous solution at a concentration of about 1 mg CNP/mL.
Clause 5. The composition of any one of clauses 1-4, wherein the CNP displays fluorescence in the blue, green, red, and infrared spectra.
Clause 6. The composition of any one of clauses 1-5, wherein the CNP has amphiphilic properties.
Clause 7. A method for reversible encapsulation of a molecule within the CNP of any one of clauses 1-6, the method comprising:
   contacting the open form CNP with a molecule to be encapsulated;
   converting the open form CNP to the closed form CNP with the molecule encapsulated therein by adjusting the pH to about 7.0 or greater; and
   releasing the encapsulated molecule from the closed form CNP by converting back to the open form CNP at a pH of about 6.8 or lower.
Clause 8. The method of any one of clauses 1-6, wherein the molecule is one or more of a small molecule pharmaceutical, a protein, a peptide, a nucleic acid, a single strand DNA, a double strand DNA, an RNA, an siRNA, an oligonucleotide, a gene, a gene fragment, an imaging agent, a lanthanide, or a combination thereof.
Clause 9. The method of any one of clauses 1-8, wherein the molecule is no more than about 500 kDa in size.
Clause 10. A composition comprising a water-soluble carbon nanoparticle (CNP) having an encapsulated molecule therein, the CNP comprising a plurality of graphene sheets having a plurality of carboxylic acid groups and hydroxyl groups and interconvertible open and closed forms; the open form having a size of about 40 nm to 200 nm in breadth and length in open sheet form; the closed form having a diameter of about 50 nm to 80 nm; wherein the closed form can be converted to the open form at a pH of about 6.8 or lower; and the open form can be converted to the closed form at a pH of about 7.0 or greater.
Clause 11. The composition of clause 10, wherein the carboxylic acid groups comprise at least about 25-35% of the total mass of the CNP.
Clause 12. The composition of clause 10 or 11, wherein the hydroxyl groups comprise at least about 15% of the total mass of the CNP.
Clause 13. The composition of any one of clauses 9-12, wherein the encapsulated molecule is an imaging agent or a therapeutic agent.
Clause 14. The composition of any one of clauses 9-13, wherein the encapsulated molecule is a therapeutic agent for the treatment of cancer.
Clause 15. The composition of any one of clauses 9-14, wherein the encapsulated molecule is one or more of a small molecule pharmaceutical, a protein, a peptide, a nucleic acid, a single strand DNA, a double strand DNA, an RNA, an siRNA, an oligonucleotide, a gene, a gene fragment, an imaging agent, a lanthanide, or a combination thereof.
Clause 16. The composition of any one of clauses 9-15, wherein the molecule is no more than about 500 kDa in size.
Clause 17. The composition of any one of clauses 9-16, further comprising one or more biomolecules or divalent metals for targeted delivery of the CNP with the encapsulated molecule to a cell, tissue, brain, or organ.
Clause 18. The composition of any one of clauses 9-17, wherein the biomolecule is a protein, a receptor, an aptamer, a ligand, or an antibody; and the divalent metal is manganese (Mn).
Clause 19. A method for treating a subject with cancer, the method comprising:
   delivering to the subject a pH-sensitive water-soluble carbon nanoparticle (CNP) having an encapsulated molecule therein,
   wherein the CNP comprises a plurality of graphene sheets having a plurality of carboxylic acid groups and hydroxyl groups and interconvertible open and closed forms; the open form having a size of about 40 nm to 200 nm in breadth and length in open sheet form; the closed form having a diameter of about 50 nm to 80 nm; wherein the closed form can be converted to the open form at a pH of about 6.8 or lower; and the open form can be converted to the closed form at a pH of about 7.0 or greater; and
   wherein the encapsulated molecule is released from the CNP at a pH of 6.8 or lower after delivery to the subject.
Clause 20. The method of clause 19, wherein delivering comprises parenteral administration, oral administration, or inhalation.
Clause 21. The method of clause 19 or 20, wherein the molecule is one or more of a small molecule pharmaceutical, a protein, a peptide, a nucleic acid, a single strand DNA, a double strand DNA, an RNA, an siRNA, an oligonucleotide, a gene, a gene fragment, an imaging agent, a lanthanide, or a combination thereof.
Clause 22. The method of any one of clauses 19-21, wherein the small molecule pharmaceutical is paclitaxel.
Clause 23. The method of any one of clauses 19-22, wherein the cancer is breast, ovarian, lung, bladder, prostate, melanoma, esophageal, stomach, other solid tumor cancers, combinations thereof.
Clause 24. The method of any one of clauses 19-23, wherein the subject has glioblastoma.
Clause 25. The method of any one of clauses 19-24, wherein the CNP further comprises one or more biomolecules or divalent metals for targeted delivery of the CNP with the encapsulated molecule to a cell, tissue, organ, or organ system.
Clause 26. The method of any one of clauses 19-25, wherein the biomolecule is a protein, a receptor, an aptamer, a ligand, or an antibody; and the divalent metal is manganese (Mn).
Clause 27. The method of any one of clauses 19-26, wherein the molecule is delivered across the blood brain barrier (BBB) of the subject.
Clause 28. A method for producing a pH-sensitive water-soluble carbon nanoparticle (CNP), the method comprising:
   treating a material comprising one or a combination of wood, charcoal, low grade coal, or carbonized plant biomass with a dilute acidic solution to form a mixture of components including insoluble material and a second solution;
   separating the second solution from the insoluble material;
   neutralizing the second solution to form a precipitate; and
   separating the precipitate from the neutralized second solution;
   wherein the precipitate comprises the CNP, the CNP comprising a plurality of graphene sheets having a plurality of carboxylic acid groups and hydroxyl groups and interconvertible open and closed forms; the open form having a size of about 40 nm to 200 nm in breadth and length in open sheet form; the closed form having a diameter of about 50 nm to 80 nm; wherein the closed form can be converted to the open form at a pH of about 6.8 or lower; and the open form can be converted to the closed form at a pH of about 7.0 or greater.
Clause 29. The method of clause 28, wherein the dilute acidic solution is diluted HNO₃.
Clause 30. The method of clause 28 or 29, wherein the carboxylic acid groups comprise at least about 25-35% of the total mass of the CNP.
Clause 31. The method of any one of clauses 28-30, wherein the hydroxyl groups comprise at least about 15% of the total mass of the CNP.

### EXAMPLES

### Example 1

### CNP Production Method

Organic waste such as hay, wood shavings, and sugarcane is carbonized in a furnace in an inert atmosphere of argon at a temperature ranging from about 500° C. to 700° C. The carbonized organic waste is washed several times with acetone or toluene to remove polyaromatic hydrocarbon (PAH) impurities. The carbonized material free from PAH impurities is then treated with dilute nitric acid (4 N) in an ice bath. This leaches out the graphene oxide (GO) and reduced graphene oxide (rGO), which are naturally present in the carbonized material (e.g., coke, coal, wood, charcoal, etc.). The dilute HNO₃ used here serves the purpose of leaching out GO and transforming the rGO to GO for solubility purposes. Hence, the dilute HNO₃ is not used as just any conventional oxidizing agent. This method is different from the Hummers method, which uses very strong oxidizing agents such as a mixture of concentrated HNO₃, H₂SO₄, and solid KMNO₄, to oxidize the graphite to GO.

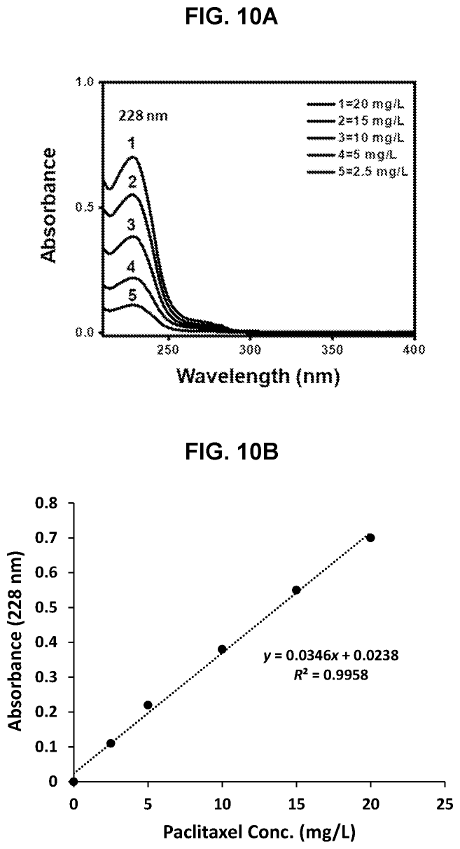

After 24 hours of treatment with the dilute nitric acid, any nitric acid left with the solid carbon is completely dried under a warm water bath not exceeding a temperature of 50° C. The dried carbon is washed with cold distilled water (ddH₂O) several times to remove the leftover nitric acid present in the powdered carbon. The GO and rGO that is leached out of the carbonized material by treating with the dilute HNO₃ is then treated with NaOH (5% to 10% concentration) to extract GO and rGO, changing the GO to a sodium salt of GO, which is soluble in water. The sodium salt or derivative of GO is treated with dilute HCl (0.001 N to 0.0006 N) resulting in the separation of sodium salt-free GO. The GO particles extracted following treatment with the dilute HCl is in the size range of about 70 nm to about 120 nm, as measured by dynamic light scattering (DLS) (FIG. 8A-B). Based on this size range, the generated GO particles are considered carbon nanoparticles (CNPs).

The CNPs extracted from the original source organic waste, coke, coal source, etc. are comprised of several CNPs that vary in size ranging from about 20 nm to about 120 nm. Furthermore, each of these CNPs have a different number of functional hydrophilic groups attached such as carboxylic acids and hydroxyl groups. These CNPs with varying size and functional groups react differently when treated with alkali or base to neutralize the peripheral functional groups. The way in which they react depends on the difference in the pKₐ values of the carboxylic acid groups, as there will be several pKₐ values due to the presence of varying amounts of carboxylic acid groups. However, it is possible to use a specific pH to separate out CNPs with different pKₐ values from the bulk mixture of CNPs.

Treatment of the CNP mixture with alkali results in the formation of ionic carboxylate formed with sodium ion, creating a soluble form of CNP that is in an open form, shown in FIG. 1. This solubilized form of CNP is centrifuged and the filtrate is treated to a preferential pH, which results in separation of a specific form of CNP that is susceptible to that specific pH.

The acidification of the filtrate obtained after treating the CNP mixture with NaOH is done by slowly treating it with very dilute HCl (0.001 N to 0.0006 N) to bring to a desired pH level such as pH 6.5. This acidification process isolates CNPs that open at a preferred pH of 6.5 from the rest of the CNP mixture. If the NaOH-treated CNP solution is subjected to acidification using a narrow pH such as pH 6.8, it will separate and isolate the CNPs susceptible to that specific pH from other CNPs in the mixture. Re-precipitation by slightly lowering the pH to 6.5, for example, completely separates the CNP on precipitation that will operate only in the applied narrow pH range (pH 6.5-6.8).

The resultant solution after very slow treatment with very dilute HCl (0.001N to 0.0006N) to get to a desired pH of 6.5 or 6.7, for example, is left in a cold-water bath for 24-48 hours to slowly precipitate the CNP that is susceptible to pH 6.5 or 6.7. This would take 2-3 days to precipitate CNP in open format for a desired pH of 6.5. This process removes the CNP that opens at the desired pH from the mixture of CNPs. These CNPs can then be used to encapsulate cargo (i.e., a drug) as they are in open form.

The treatment of the CNP mixture after NaOH extraction, followed by slow acidification with very dilute acid at a desired pH, is a unique method for separating and isolating CNPs which are pH susceptible that can then be used to enclose and release drugs in any desired/specific pH.

The open form CNP product was placed in ddH₂O with a pH of 6.5 or any pH limit desired, such as pH 6.3. The CNP on digestion precipitated resulting in CNP which closes at pH 6.5. Therefore, this CNP will operate in an open form at pH 6.8 and in a closed form at pH 6.5.

Therefore, the described methods allow for the preparation of CNPs which open and close at specific pH values, and the required pH will need to be used for opening and closing of the CNP. For example, if a CNP is desired that is open at pH 6.7 but remains closed at pH 6.3, a ddH₂O solution at pH 6.7 has to first be prepared and added to this CNP, which is then allowed to stand (i.e., digest) in a cold-water bath. This results in CNP soluble at pH 6.7, which is centrifuged and filtered. To the filtrate, very dilute HCl acid is added, resulting in a pH drop down to 6.3, which closes the CNP. As a result, a CNP product is formed that opens at pH 6.7 and closes at pH 6.3.

Shown below in Table 1 are data demonstrating the pH release profile of CNP encapsulated payload in a pH range from 6.5 to 7.0 over a 72 hr time period. The data clearly point out that the release of the payload inside the CNP is much faster at pH 6.5 than that at pH 7.0 in the initial hour. Increasing hours will have some interaction with the bulk water present as medium whose pKₐ value remains close in range to the pH under study. The difference in pH is narrowed due to the dissociation of water. However, the initial hour is crucial where the payload at pH 6.5 is released more than double in quantity compared to payload at pH 7.0, and this is crucial for the selective release of drugs at sites with lower pH values.

TABLE 1
pH Release Profile of CNP Payload Over Time (cumulative mg/mL)
Time (hours)

| pH | 1 | 2 | 4 | 7 | 10 | 16 | 24 | 48 | 72 |
|---|---|---|---|---|---|---|---|---|---|
| 6.5 | 68 | 88 | 131 | 150 | 172 | 185 | 210 | 236 | 262 |
| 6.8 | 32 | 53 | 91 | 102 | 145 | 160 | 196 | 215 | 220 |
| 7.0 | 30 | 43 | 73 | 84 | 141 | 150 | 160 | 205 | 220 |

### Example 2

### Structural Characteristics of CNPs

FIG. 1 and FIG. 2 show SEM images confirming the open and closed forms of CNPs, respectively. To confirm the open state of CNPs, 1 mg of CNP is treated with ddH₂O at pH 6.5. This is sonicated for 2-3 minutes. A drop of this solution is used for SEM analysis to show the open state of CNP (FIG. 1). To confirm the closed state of CNPs, 1 mg of CNP is treated with ddH₂O at pH 7.5. This is sonicated for 2-3 minutes. A drop of this solution is used for SEM analysis to show the closed state of CNP (FIG. 2).

As shown in FIG. 8A-B, size distribution studies using DLS at pH 7 demonstrate that the mean size maximum peak is at 90 nm. Furthermore, more than 90% of the particles fall within the range of 70-120 nm. At pH 7, the majority of the CNPs are in the closed form.

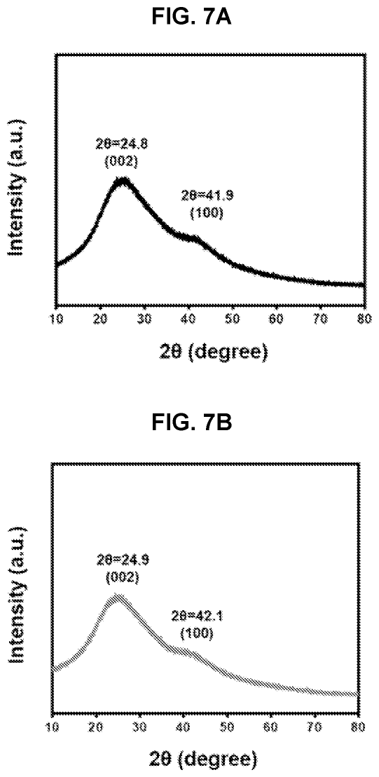

The Raman spectrum of CNP from FIG. 5 shows a strong peak at 1577 cm⁻¹, corresponding to the in-plane stretching vibration of the sp 2 C—C bonds (G band) within the ordered graphitic layers. Another vibration at 1356 cm⁻¹ (D band; defect band) is related to defects in the graphene structure of CNP (FIG. 5). The Raman spectrum confirms the presence of GO and rGO. As these are 2D materials, the in and out plane stretching are responsible for origin of the G and D bands.

### UV-Vis Spectra of CNPs

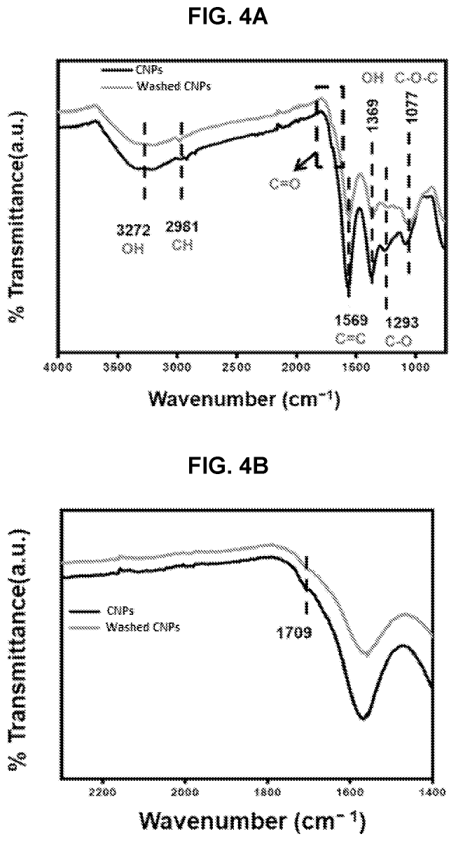

A UV-Vis spectrum of CNPs in water shows a single broad absorption peak around 267 nm (FIG. 3), characteristic of electron transition in the polyaromatic groups of GO.

### Zeta Potential of CNPs

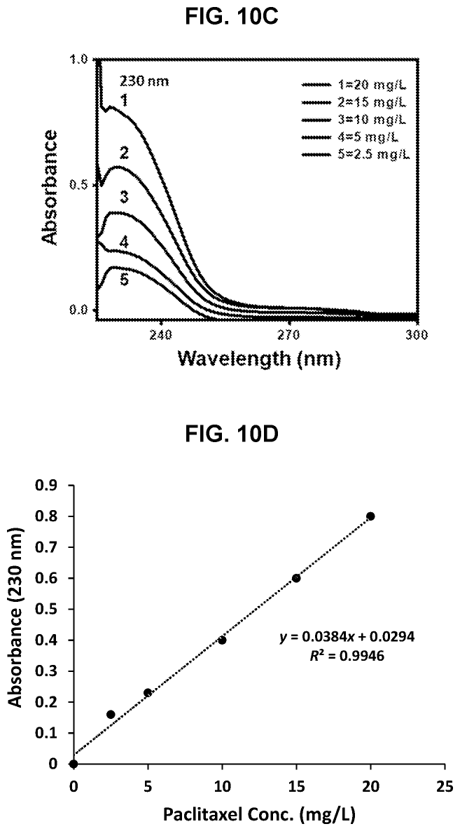

The Zeta potential value helps in determining the stability of particles. Zeta potential values greater than ±60 mV indicate excellent stability, but particles with values between +10 to −10 mV will experience rapid agglomeration. The Zeta potential value of CNP is −34.4 mV (FIG. 9; Table 2), which implies good stability of the particles. As the charge is negative, the carboxylic (—COO⁻) and hydroxyl (—O⁻) groups are responsible for the high Zeta potential values. The high negative value also suggests that the CNP is electrostatically stable in pH 7 and does not agglomerate.

TABLE 2
Zeta Potential of CNPs

| | |
|---|---|
| Zeta Potential (mV) | −34.4 |
| Zeta Deviation (mV) | 9.47 |
| Conductivity (mS/cm) | 0.0199 |
| Result Quality | Good |
| Peak 1 | −34.4 |
| Area (%) | 100 |
| Std Dev (mV) | 9.47 |

### ATR-IR Spectra of CNPs and Washed CNPs

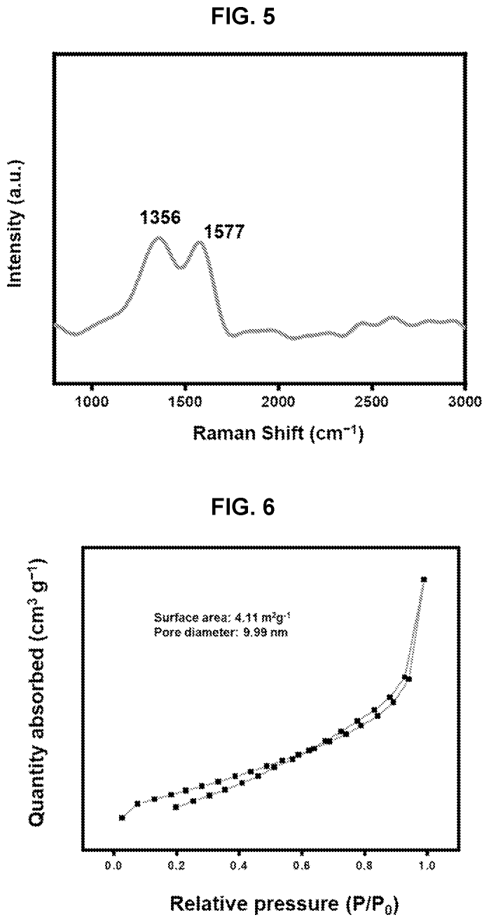

Attenuated total reflection infrared (ATR-IR) spectra of CNPs and washed CNPs are shown in FIG. 4A-B. FIG. 4A shows spectra of CNP and washed CNP. The broad peaks at 3272 cm⁻¹ and 2981 cm⁻¹ correspond to the O—H stretching of the hydroxyl group and the C—H stretching of the graphene sheet, respectively. The presence of the carboxylic group is confirmed by the peak at 1709 cm⁻¹, which corresponds to C═O stretching in COOH. Zoomed-in ATR-IR spectra show the presence of an acid functional group (FIG. 4B). Peaks corresponding to C═C stretching, O—H bending, and C—O stretching are assigned at 1569 cm⁻¹, 1369 cm⁻¹, and 1293 cm⁻¹, respectively. The peak at 1077 cm⁻¹ confirms the presence of the epoxy group. Washing was performed to remove traces of NaCl. Notably, there were no significant changes in the IR spectra when the CNPs were washed. The zoomed-in spectra clearly show the presence of the COOH group (FIG. 4B). A summary of the ATR-IR data is shown below in Table 3.

TABLE 3
ATR-IR spectra data of CNPs and washed CNPs (FIG. 4A-B)

| Wavenumber | Nature | Functional |
|---|---|---|
| (cm⁻¹) | of peak | group |
| 3272 | Strong, broad | O—H stretching, alcohol |
| 2981 | Weak | C—H stretching, alkane |
| 1709 | Weak | C═O stretching (—COOH group) |
| 1569 | Strong | C═C stretching |
| 1369 | Strong | O—H bending alcohol |
| 1293 | Weak | C—O stretching |
| 1077 | Medium | C—O—C epoxy group |

### Powder X-ray Diffraction of CNPs

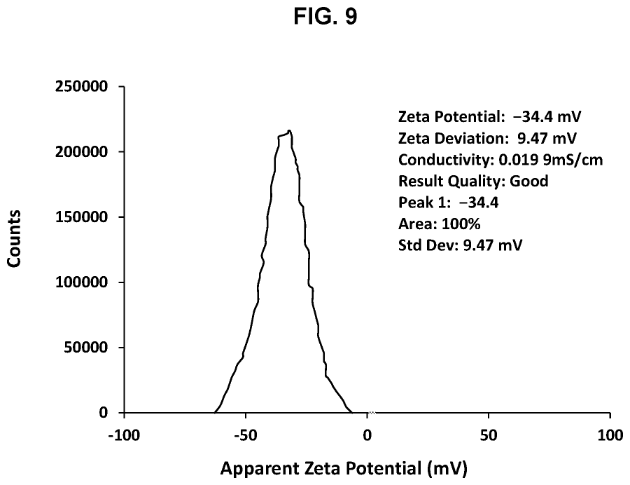

CNPs were washed in two different ways to completely remove NaCl and powder X-ray diffraction (PXRD) was recorded. The two PXRD patterns of washed CNPs show that the CNPs are amorphous in nature (FIG. 7A-B). The peaks at 20=24.8 (FIG. 7A) and 20=24.9 (FIG. 7B) appear due to the hkl=(002) plane and peak at 20=41.9 (FIG. 7A) and 20=42.1 (FIG. 7B) for the hkl=(100) plane, respectively. These two types of peaks reveal reduced graphene oxide peaks.

For FIG. 7A, the protocol was as follows: 1 g of CNPs was leached with NaOH, precipitated by HCl and the precipitate was washed using whatman41 filter paper. The washing was performed by first washing with 30 mL cold water, followed by a second wash with 1 mL EtOH and 29 mL of water mixture. A third wash was performed with 5 mL EtOH and 25 mL water mixture, and finally followed with 10 mL of EtOH with 20 ml water to yield 800 mg.

For FIG. 7B, the protocol was as follows: 5 g of CNPs was leached with NaOH, precipitated by HCl and the precipitate was collected using Whatman filter paper. The washing was performed by first washing with 50 mL of ice-cold water; followed by a second wash with 50 mL of ice-cold water. A third wash was performed with 50 mL of ice-cold water, and finally followed with 30 mL of EtOH to yield 4.2 g.

Whether the material is crystalline or amorphous can be determined using the PXRD patterns. Crystalline materials normally give sharp peaks, but amorphous materials give broader peaks. Here, broad peaks observed for the CNPs indicate that the material is amorphous.

Planes hkl=(002) and (100) are crystallographic plans. They are represented as (hkl) and are also called miller indices for the plan. If Bragg's law equation is used, they are represented as nλ=2 dₕₖₗ sin θ, where n=diffraction order, λ=wavelength of radiation, dₕₖₗ=inter planner or atomic spacing, and θ=angle between incident light and surface of the plan.

### Brunauer-Emmett-Teller (BET) Surface Area Analysis of CNPs

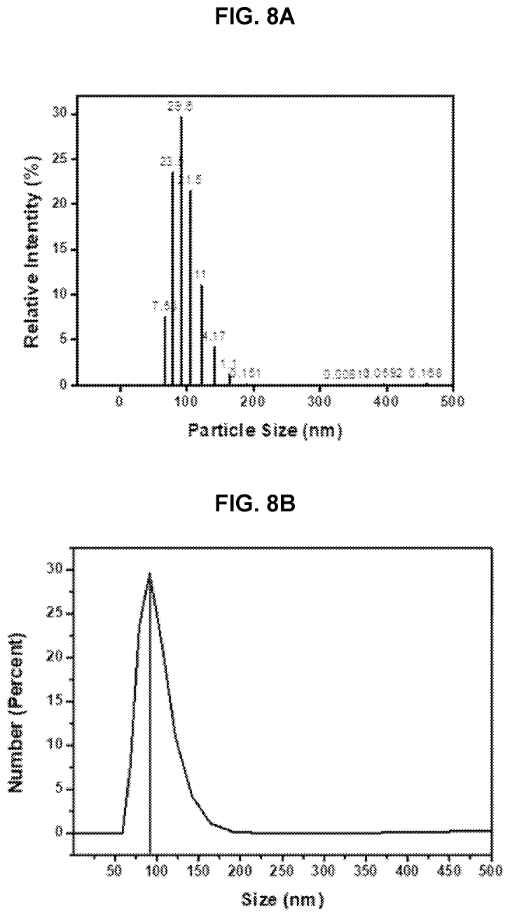

The surface area and pore size distribution of CNPs were measured using BET and BJH methods. The calculated BET surface area and pore diameter of prepared CNPs are 4.11 m²g⁻¹ and 9.99 nm, respectively (FIG. 6). This BET analysis indicates the specific surface area, pore size, and pore volume of the material.

### Example 3

### In Vitro Paclitaxel Studies

### Standard Curve for Paclitaxel Studies

To determine a standard curve for paclitaxel, a 100 PPM (mg/L) paclitaxel solution was first generated by dissolving 10 mg paclitaxel in 100 mL of EtOH. From this 100 PPM stock solution, 10 mL of 20, 15, 10, 5, and 2.5 PPM solutions were prepared by dilution and UV-Vis was measured to generate the standard curve for paclitaxel in EtOH. Similarly, paclitaxel solution in dichloromethane (DCM) was made and UV-Vis spectra were measured to create the standard curve for paclitaxel in DCM solvent. After measuring UV-Vis spectra, absorbances were obtained for the different concentration solutions. See Table 4. After plotting the absorbance values along the y-axis with respect to the concentrations of the solutions along the x-axis, a straight line in each case was made, validating Lambert-Beers law. The unknown concentration of the paclitaxel drug could then easily be obtained from the absorbance value (OD value) of these plots.

TABLE 4
Standard Curves for Paclitaxel in Ethanol and Dichloromethane

| Concentration (mg/L) | Absorbance (EtOH) | Absorbance (DCM) |
|---|---|---|
| 0 | 0 | 0 |
| 2.5 | 0.11 | 0.16 |
| 5 | 0.22 | 0.23 |
| 10 | 0.38 | 0.4 |
| 15 | 0.55 | 0.6 |
| 20 | 0.7 | 0.8 |

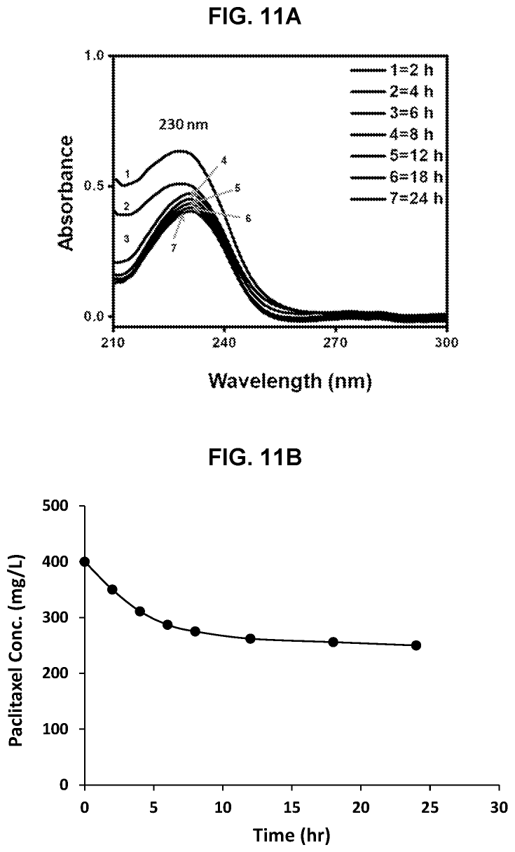

FIG. 10A-D show the standard curves that were generated for paclitaxel in both ethanol and DCM solvents. FIG. 10A shows UV-Vis spectra of paclitaxel at varying concentrations in ethanol. FIG. 10B shows a standard curve for absorbance vs. concentration of paclitaxel in ethanol. FIG. 10C shows UV-Vis spectra of paclitaxel at varying concentrations in DCM. FIG. 10D shows a standard curve for absorbance vs. concentration of paclitaxel in DCM.

### Absorption Kinetic Studies for Paclitaxel

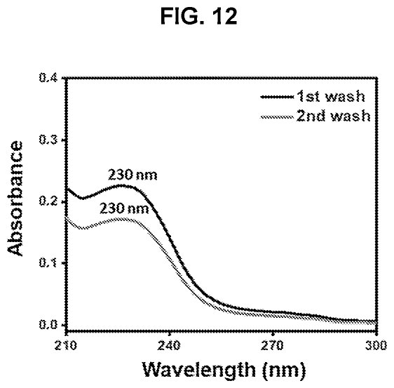

Absorption kinetic studies of paclitaxel are shown in FIG. 11A-B. FIG. 11A shows UV-Vis spectra of paclitaxel in the presence of CNPs over different time intervals, while FIG. 11B shows a plot of the concentration of paclitaxel in solution vs. time. The curve of FIG. 11B was determined from absorption kinetics study. The concentration of paclitaxel in solution measured using UV-Vis is plotted for different time intervals. The measured concentration is plotted along the y-axis with respect to time along the x-axis. The graph of FIG. 11B reveals that with increasing time, the concentration of paclitaxel in solution decreases and reaches equilibrium by 24 hours. A summary of the absorption capacity of CNPs after 24 hours is shown below in Table 5.

TABLE 5
Absorption capacity of CNPs after 24 hours

| Initial | Initial | Paclitaxel present | Paclitaxel | Absorption |
|---|---|---|---|---|
| [paclitaxel] | [CNPs] | in soln. after | absorbed after | capacity |
| (mg/L) | (g/L) | 24 hr (mg/L) | 24 hr (mg/L) | (mg/g) |
| 400 | 0.4 | 250.6 | 149.4 | 373 |

### Leaching Study for Paclitaxel

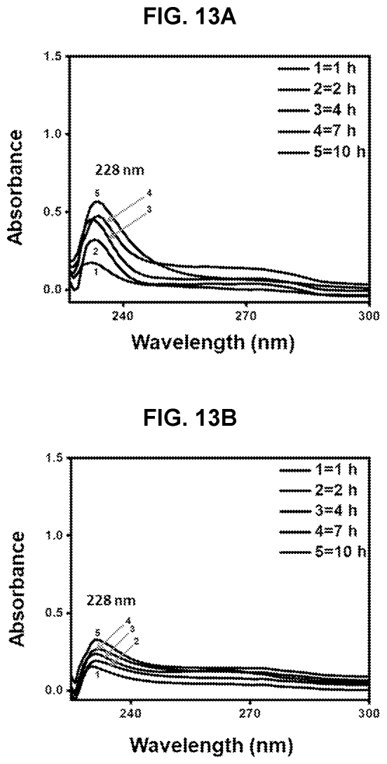

After 24 hr of aging, the paclitaxel mixture was filtered, and the residue was dried completely. The residue was then washed with 10 mL of EtOH, and the UV-Vis spectrum of the wash solvent was recorded to estimate the free, unbound paclitaxel present outside the CNPs, which are either freely bounded or not absorbed in the CNPs (FIG. 12). The washing process was initially performed twice to show such surface contamination, but a third washing cycle did not show any contamination, indicating that the product was free from external absorbed drug. The uptake capacity of CNPs in mg/g scale was calculated and the absorption capacity of CNPs was found to be 304 mg/g for paclitaxel. A summary of the absorption capacity of CNPs after washing is shown below in Table 6.

TABLE 6
Absorption capacity of CNPs after washing

| | | Paclitaxel | Paclitaxel | |
|---|---|---|---|---|
| Initial | Initial | absorbed | in wash | Absorption |
| [paclitaxel] | [CNPs] | after 24 hr | solvent | capacity |
| (mg/L) | (g/L) | aging (mg/L) | (mg/L) | (mg/g) |
| 400 | 0.4 | 149.4 | 27.8 | 304 |

### Paclitaxel Release Profile at Various pH Values

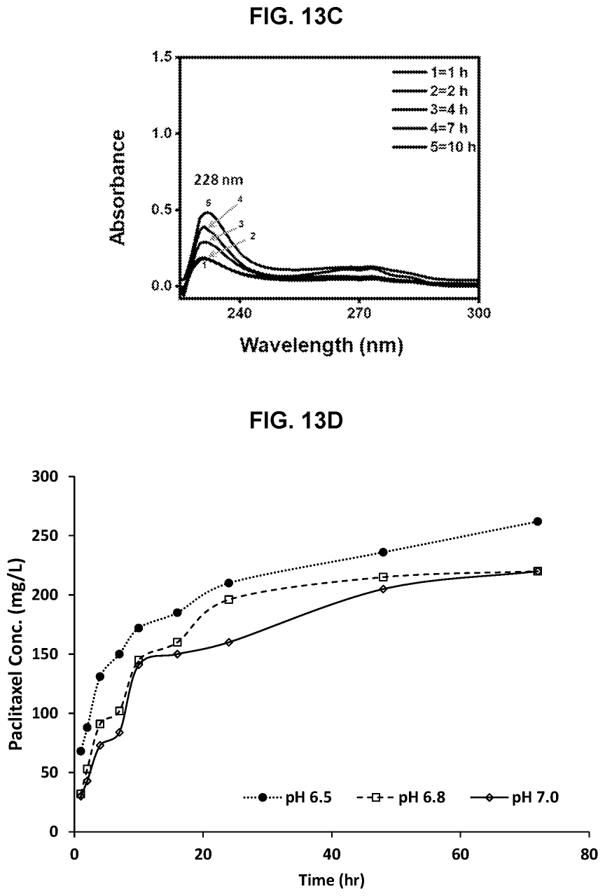

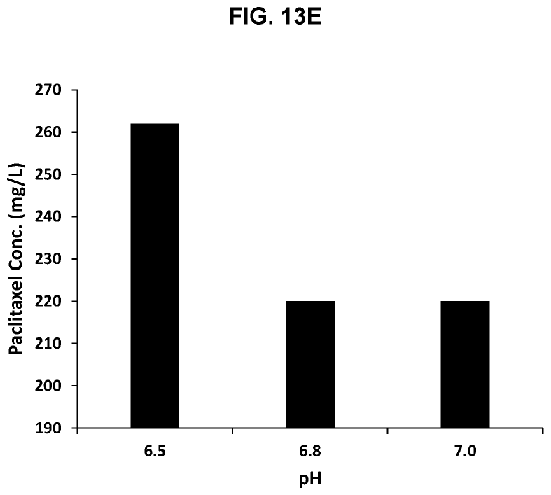

The concentration of paclitaxel released at different pH from encapsulated CNPs at different time intervals was calculated. FIG. 13A-E show the paclitaxel release profile by UV-Vis spectra at pH 6.5 (FIG. 13A), pH 6.8 (FIG. 13B), and pH 7.0 (FIG. 13C). FIG. 13D shows the paclitaxel release as a function of pH over time, with data for each time interval shown below in Table 7.

TABLE 7
Paclitaxel Release Profile Over Time at pH 6.5, 6.8, and 7.0 (mg/L)
Time (hr)

| pH | 1 | 2 | 4 | 7 | 10 | 16 | 24 | 48 | 72 |
|---|---|---|---|---|---|---|---|---|---|
| 6.5 | 68 | 88 | 131 | 150 | 172 | 185 | 210 | 236 | 262 |
| 6.8 | 32 | 53 | 91 | 102 | 145 | 160 | 196 | 215 | 220 |
| 7.0 | 30 | 43 | 73 | 84 | 141 | 150 | 160 | 205 | 220 |

FIG. 13E shows a histogram diagram of the paclitaxel pH release profile at 72 hr as a function of pH, with data provided below in Table 8.

TABLE 8
Paclitaxel Release as Function of pH After 72 hours

| pH | Paclitaxel Conc. (mg/L) |
|---|---|
| 6.5 | 262 |
| 6.8 | 220 |
| 7.0 | 220 |

### Example 4

### In Vivo Paclitaxel Pharmacokinetic Study

Paclitaxel (e.g., Taxol®) was procured from Adooq Biosciences LLC and CNPs were produced by SiNON Therapeutics. Acetonitrile, water for HPLC, and formic acid were all highly pure and HPLC and/or analytical grade only. DMSO, Tween 80 & Carboxy Methyl Cellulose (CMC) were used for formulation preparation.

A Shimadzu UFLC system equipped with a Quaternary pump, a refrigerated auto sampler, Thermostat controlled column oven compartment was used as front end. The detection was using a Mass quadrupole detector (AB Sciex 4500 Q Trap).

Wistar rats (male, 200-220 g) were obtained from the CPCSEA registered animal vendor. Animals were maintained at temperature of 23±2° C., relative humidity of 30% to 70%, and a 12 h dark-light cycle. All of the animals had free access to water and rodent chow at all times, and all of the experimental animals were fed under the above conditions throughout the acclimatization period. Wistar rats (n=1, Male) were randomly taken for each of the two groups. One group was administrated paclitaxel (60 mg/kg, PO) via oral gavage. The second group animal was dosed with paclitaxel encapsulated in CNPs (60 mg/kg, PO) vial oral gavage. The dose formulation for both the paclitaxel as well as the paclitaxel encapsulated CNPs was DMSO: Tween 80: 0.5% CMC: 6:12:82. All the rats were fasted for 12 hr before administration. The blood collection was done from retro-orbital plexus using an anticoagulant-coated capillary tube at 0 (pre-dose), 0.25, 0.5, 1, 2, 4, 6, 8, and 24 hr respectively following oral administration from both the rats (Group 1 and 2). At each time point, blood sample (125 μL) was collected in EDTA tubes and tubes were immediately centrifuged at 3000 rpm for 10 min to obtain the plasma. The separated plasma was frozen at −20° C. before analysis.

A conventional protein precipitation method using acetonitrile was used to extract paclitaxel from plasma samples. The biological samples were thawed at room temperature. After vortexing for 30 seconds, 100 μL of sample was taken into 1.5 mL eppendorf tubes, 600 μL of ice-cold acetonitrile was added to denature the proteins. The tubes were vortexed for 2 minutes followed by centrifugation at 5000 rpm for 10 minutes. The supernatant was transferred into HPLC glass vials with septa and were loaded into autosampler for further analysis on a mass spectrometer. The calibration curve was prepared as described above.

### Chromatographic and Mass Spectrometry Conditions

A fit to purpose LCMS-MS Bioanalytical method for quantification of paclitaxel was developed. Mobile phase used was, a channel as 0.1% formic acid in water, and a channel as acetonitrile. The flow rate was optimized to 0.4 mL/minute. A Kromasil C18 column with dimensions 150×4.6 mm, 0.5 μm was used for separation. The column temperature was maintained at 40° C. The injection volume was 20 μL. The compound-related parameters on the mass spectrometer were optimized as declustering potential, entrance potential, and exit potential of 100, 10, and 10, respectively. The collision energy was kept at 20. The source-related parameters of curtain gas and collision gas were optimized to 30 and 8, respectively. The ionspray voltage was kept at 5500 and ion source gas 1 and ion source gas 2 were kept at 40° C. each, respectively. The source temperature was optimized to 500° C. The Analyst software was used for mass data acquisition.

A linearity curve was developed using 7 points calibration samples from 0.0312 to 4.0 μg/mL and a Regression coefficient of 0.99 was obtained. All of the seven calibration points passed with accuracy of 80-120% and a quality control sample also passed with close to 100% accuracy.

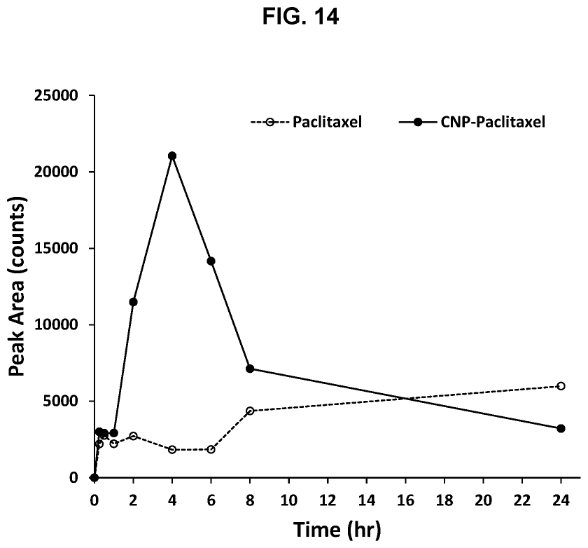

Consistent with previous results where the plasma levels of paclitaxel in wild-type mice receiving the drug by the oral route remained very low, i.e., the plasma levels hardly exceeded the 0.1 mM (85 ng/mL) level, which is considered of therapeutic relevance, very low concentrations of paclitaxel were observed. The concentration of paclitaxel after oral administration of paclitaxel and CNP encapsulated paclitaxel in Wistar rats at the majority of the timepoints showed concentrations below the detection limit, i.e., concentrations <0.312 μg/mL. However, a trend in concentrations at a few time points for CNP encapsulated paclitaxel-treated rats versus paclitaxel-treated rats was observed. Hence, the peak area counts and plotted graph for Peak Areas-time curves in rats receiving oral paclitaxel and CNP encapsulated paclitaxel (60 mg/kg) is shown in FIG. 14. The paclitaxel Peak Area versus timepoints data are tabulated in Table 9.

TABLE 9
Peak Area Over Time for Paclitaxel or CNP-
Encapsulated Paclitaxel in Wistar Rats

| Time (hr) | Paclitaxel | CNP-Paclitaxel |
|---|---|---|
| 0.25 | 2200 | 3000 |
| 0.50 | 2753 | 2914 |
| 1.00 | 2213 | 2918 |
| 2.00 | 2720 | 11490 |
| 4.00 | 1832 | 21044 |
| 6.00 | 1845 | 14158 |
| 8.00 | 4361 | 7126 |
| 24.00 | 5991 | 3214 |

The plasma concentrations following oral administration of paclitaxel were below quantitation limits of 0.0312 μg/mL. However, based on analyte peak area (counts), it was observed that CNP encapsulated paclitaxel appears to deliver greater concentration than paclitaxel, thus indicating improved bioavailability.

What is claimed:

1. A composition comprising graphene oxide nanoparticles (CNP) comprising a plurality of graphene sheets having a plurality of carboxylic acid groups and hydroxyl groups and interconvertible open and closed forms; the open form having a size of about 40 nm to 200 nm in breadth and length in open sheet form; the closed form having a diameter of about 50 nm to 80 nm; wherein the closed form can be converted to the open form at a pH of about 6.8 or lower; and the open form can be converted to the closed form at a pH of about 7.0 or greater.

2. The composition of claim 1, wherein the carboxylic acid groups comprise at least about 25-35% of the total mass of the CNP.

3. The method of claim 1, wherein the hydroxyl groups comprise at least about 15% of the total mass of the CNP.

4. The composition of claim 1, wherein the CNP has a solubility in aqueous solution at a concentration of about 1 mg CNP/mL.

5. The composition of claim 1, wherein the CNP displays fluorescence in the blue, green, red, and infrared spectra.

6. The composition of claim 1, wherein the CNP has amphiphilic properties.

7. A method for reversible encapsulation of a molecule within the CNP of claim 1, the method comprising:
contacting the open form CNP with a molecule to be encapsulated;
converting the open form CNP to the closed form CNP with the molecule encapsulated therein by adjusting the pH to about 7.0 or greater; and
releasing the encapsulated molecule from the closed form CNP by converting back to the open form CNP at a pH of about 6.8 or lower.

8. The method of claim 7, wherein the molecule is one or more of a small molecule pharmaceutical, a protein, a peptide, a nucleic acid, a single strand DNA, a double strand DNA, an RNA, an siRNA, an oligonucleotide, a gene, a gene fragment, an imaging agent, a lanthanide, or a combination thereof.

9. The method of claim 8, wherein the molecule is no more than about 500 kDa in size.

10. A composition comprising a water-soluble carbon nanoparticle (CNP) having an encapsulated molecule therein, the CNP comprising a plurality of graphene sheets having a plurality of carboxylic acid groups and hydroxyl groups and interconvertible open and closed forms; the open form having a size of about 40 nm to 200 nm in breadth and length in open sheet form; the closed form having a diameter of about 50 nm to 80 nm; wherein the closed form can be converted to the open form at a pH of about 6.8 or lower; and the open form can be converted to the closed form at a pH of about 7.0 or greater.

11. The composition of claim 10, wherein the carboxylic acid groups comprise at least about 25-35% of the total mass of the CNP.

12. The method of claim 10, wherein the hydroxyl groups comprise at least about 15% of the total mass of the CNP.

13. The composition of claim 10, wherein the encapsulated molecule is an imaging agent or a therapeutic agent.

14. The composition of claim 10, wherein the encapsulated molecule is a therapeutic agent for the treatment of cancer.

15. The composition of claim 10, wherein the encapsulated molecule is one or more of a small molecule pharmaceutical, a protein, a peptide, a nucleic acid, a single strand DNA, a double strand DNA, an RNA, an siRNA, an oligonucleotide, a gene, a gene fragment, an imaging agent, a lanthanide, or a combination thereof.

16. The composition of claim 10, wherein the molecule is no more than about 500 kDa in size.

17. The composition of claim 10, further comprising one or more biomolecules or divalent metals for targeted delivery of the CNP with the encapsulated molecule to a cell, tissue, brain, or organ.

18. The composition of claim 17, wherein the biomolecule is a protein, a receptor, an aptamer, a ligand, or an antibody; and the divalent metal is manganese (Mn).

19. A method for treating a subject with cancer, the method comprising:
delivering to the subject a pH-sensitive water-soluble carbon nanoparticle (CNP) having an encapsulated molecule therein,
wherein the CNP comprises a plurality of graphene sheets having a plurality of carboxylic acid groups and hydroxyl groups and interconvertible open and closed forms; the open form having a size of about 40 nm to 200 nm in breadth and length in open sheet form; the closed form having a diameter of about 50 nm to 80 nm; wherein the closed form can be converted to the open form at a pH of about 6.8 or lower; and the open form can be converted to the closed form at a pH of about 7.0 or greater; and
wherein the encapsulated molecule is released from the CNP at a pH of 6.8 or lower after delivery to the subject.

20. The method of claim 19, wherein delivering comprises parenteral administration, oral administration, or inhalation.

21. The method of claim 19, wherein the molecule is one or more of a small molecule pharmaceutical, a protein, a peptide, a nucleic acid, a single strand DNA, a double strand DNA, an RNA, an siRNA, an oligonucleotide, a gene, a gene fragment, an imaging agent, a lanthanide, or a combination thereof.

22. The method of claim 21, wherein the small molecule pharmaceutical is paclitaxel.

23. The method of claim 19, wherein the cancer is breast, ovarian, lung, bladder, prostate, melanoma, esophageal, stomach, other solid tumor cancers, combinations thereof.

24. The method of claim 19, wherein the subject has glioblastoma.

25. The method of claim 19, wherein the CNP further comprises one or more biomolecules or divalent metals for targeted delivery of the CNP with the encapsulated molecule to a cell, tissue, organ, or organ system.

26. The method of claim 25, wherein the biomolecule is a protein, a receptor, an aptamer, a ligand, or an antibody; and the divalent metal is manganese (Mn).

27. The method of claim 19, wherein the molecule is delivered across the blood brain barrier (BBB) of the subject.

28. A method for producing a pH-sensitive water-soluble carbon nanoparticle (CNP), the method comprising:
treating a material comprising one or a combination of wood, charcoal, low grade coal, or carbonized plant biomass with a dilute acidic solution to form a mixture of components including insoluble material and a second solution;
separating the second solution from the insoluble material;
neutralizing the second solution to form a precipitate; and
separating the precipitate from the neutralized second solution;
wherein the precipitate comprises the CNP, the CNP comprising a plurality of graphene sheets having a plurality of carboxylic acid groups and interconvertible open and closed forms; the open form having a size of about 40 nm to 200 nm in breadth and length in open sheet form; the closed form having a diameter of about 50 nm to 80 nm; wherein the closed form can be converted to the open form at a pH of about 6.8 or lower; and the open form can be converted to the closed form at a pH of about 7.0 or greater.

29. The method of claim 28, wherein the dilute acidic solution is diluted HNO₃.

30. The method of claim 28, wherein the carboxylic acid groups comprise at least about 25-35% of the total mass of the CNP.

31. The method of claim 28, wherein the hydroxyl groups comprise at least about 15% of the total mass of the CNP.
